(19) United States
(12) Patent Application Publication
(10) Pub. No.: US 2007/0087045 A1
(43) Pub. Date: Apr. 19, 2007

(54) Lipid carrier and method of preparing the same

(75) Inventors: Shyh-Dar Li, Miaoli County (TW); Ae-June Wang, Hsinchu (TW)

Correspondence Address: BIRCH STEWART KOLASCH & BIRCH, PO BOX 747, FALLS CHURCH, VA, 22040-0747, US

(73) Assignee: INDUSTRIAL TECHNOLOGY RESEARCH INSTITUTE

(21) Appl. No.: 11/249,326

(22) Filed: Oct. 14, 2005

(51) Int. Cl. A61K 48/00; A61K 9/127; C12N 15/88

(52) U.S. Cl. 424/450; 435/458; 514/44; 977/907

(57) ABSTRACT

A lipid carrier. The carrier includes a lipid based particle comprising a cationic lipid, a cholesterol, a neutral phospholipid, and a neutral lipid, wherein the cationic lipid is about 100 parts by weight, the cholesterol is about 25˜100 parts by weight, the neutral phospholipid is about 25˜100 parts by weight, and the neutral lipid is about 25˜150 parts by weight. The invention also provides a method of preparing the lipid carrier.

### BACKGROUND

The invention relates to a lipid carrier, and more specifically to a pH-sensitive and serum-resistant lipid carrier and a method of preparing the same.

Gene therapies have gained increasing popularity in medical treatment, especially for hereditary or acquired diseases, such as primary immune deficiencies and cancers. Gene therapy has entered in vivo experiment and human clinical trial from vitro experiment in the past fifteen years. Although the clinical trials of gene therapy are still in phase I or II, gene therapy appears promising in the future. Generally speaking, carriers delivering gene drugs are divided into viral and non-viral two types, wherein viral carriers are used in 85% of clinical trials currently.

Nevertheless, in September 1999, a teenager with Ornithine Transcarbamylase Deficiency (OTC) died during treatment with gene therapy at Pennsylvania Medicine Center. In February of the following year, the FDA stopped all clinical trials of gene therapy in U.S.A. In addition, a child with X-linked Severe Combined Immune Deficiency (X-SCID) developed leukemia after treatment with gene therapy in September 2002 in France. Unfortunately, a second leukemia case occurred in 2003 in the same clinical trial. Main virulence factors were determined to be immunogenecity and toxicity from viral carriers. Thus, development of a non-viral gene trnasfection system became a critical point in gene therapy. Currently, non-viral carriers include lipid and polymer based carriers, with lipid carriers discussed herein.

Cationic lipid carriers, most commonly used, present the highest transfection efficiency among non-viral carriers. There are several in vitro reagents available presently, such as Lipofectin and Lipofectamine from Life Technologies, applicable in gene and protein researches. Besides high transfection efficiency, cationic lipid carriers also provide the advantage of endosomolysis activity and high biocompatibility. Nevertheless, the cationic lipid and DNA complex generally exhibit a diameter exceeding 500 nm, and also are unstable in serum. Thus, the complex is mainly used in vitro study or for local injection. The DC-liposome provided by Dr. Leaf Huang was the first lipid carrier and enters clinical trial for the treatment of breast cancer by local injection. Currently, the DC-liposome is in phase II clinical trial.

In order to reduce the large particle size of cationic lipid carrier and DNA complex, Dr. Leaf Huang provides a novel gene transfection system called LPD with addition of cationic peptides, such as protamine or polylysine, to reduce the particle size and also to increase transfection efficiency. The LPD complex, however, is still unstable in serum due to the positive charge on the surface of the carrier.

To avoid serum instability and toxicity caused by cationic carriers, T. M. Allen and S. C. Semple used a neutral liposome to encapsulate DNA. The neutral liposome might reduce non-specific reaction between the carriers and proteins to enhance serum resistance. In addition, the neutral liposome might also prolong the half-life of DNA up to about 8˜24 hours (the original half-life of DNA is less than about 30 min) and also can deliver DNA to tumor sites. The carrier, however, has difficulties entering cells due to lack of the interactions between cationic carrier and the cell, resulting in low transfection efficiency. To solve the problem, target ligand is provided to trigger endocytosis by binding with a specific receptor on the cell surface, to increase DNA transfection efficiency.

Currently, most of the clinical trials by using lipid carriers are still remained in phase II. The progress is hindered by the foregoing problems such as serum instability or toxicity of the carriers. Thus, a lipid based carrier with high serum resistance, high encapsulation efficiency, and high transfection efficiency is desirable.

### SUMMARY

The invention provides a lipid carrier comprising a lipid based particle comprising a cationic lipid, a cholesterol, a neutral phospholipid, and a neutral lipid, wherein the cationic lipid is about 100 parts by weight, the cholesterol is about 25˜100 parts by weight, the neutral phospholipid is about 25˜100 parts by weight, and the neutral lipid is about 25˜150 parts by weight.

The invention also provides a method of preparing a lipid carrier, comprising the following steps. A cationic lipid, a cholesterol, a neutral phospholipid, a neutral lipid, ethanol, and water are mixed to form a lipid solution, wherein the cationic lipid is about 100 parts by weight, the cholesterol is about 25˜100 parts by weight, the neutral phospholipid is about 25˜100 parts by weight, and the neutral lipid is about 25˜150 parts by weight. Next, a drug-containing solution is added to the lipid solution to form a suspension comprising a plurality of lipid particles, wherein the drug is encapsulated into the lipid particles. Finally, the suspension is heated to form a lipid based carrier.

A detailed description is given in the following embodiments with reference to the accompanying drawings.

### BRIEF DESCRIPTION OF THE DRAWINGS

Embodiments of the invention can be more fully understood by reading the subsequent detailed description and examples with references made to the accompanying drawings, wherein:

FIG. 1 shows drug release rates of a lipid carrier in various pH conditions of the invention.

FIG. 2 shows a comparison between transfection efficiency of the lipid carriers in serum or without serum.

### DETAILED DESCRIPTION

The invention provides a lipid carrier comprising a lipid based particle comprising a cationic lipid, a cholesterol, a neutral phospholipid, and a neutral lipid, wherein the cationic lipid is about 100 parts by weight, the cholesterol is about 25˜100 parts by weight, the neutral phospholipid is about 25˜100 parts by weight, and the neutral lipid is about 25˜150 parts by weight.

The cationic lipid comprises 1,2-dioleoyloxy-3-(trimethylamino)propane (DOTAP), N-[1-(2,3-ditetradecyloxy)propyl]-N,N-dimethyl-N-hydroxyethylammonium bromide (DMRIE), N-[1-(2,3-dioleyloxy)propyl]-N,N-dimethyl-N-hydroxyethylammonium bromide (DORIE), N-[1-(2,3-dioleyloxy)propyl]-N,N,N-trimethylammonium chloride (DOTMA), 3β-[N-(N′,N′-dimethylaminoethane)carbamyl]cholesterol (DC-Chol), or dimethyldioctadecylammonium (DDAB), preferably DOTAP.

The neutral phospholipid comprises phosphatidyl choline (PC) or phosphatidyl ethanolamine (PE), wherein the phosphatidyl choline may comprise hydrogenated soy phosphatidyl choline (HSPC). The neutral lipid comprises distearoylphosphatidylethanolamine-polyethyleneglycol (DSPE-PEG).

The preferable composition of the lipid based particle is cationic lipid of about 100 parts by weight, the cholesterol about 50 parts by weight, the neutral phospholipid about 50 parts by weight, and the neutral lipid about 65 parts by weight.

The lipid based particle might be used as a drug transfection carrier when a drug is encapsulated therein, wherein the drug comprises nucleic acid drugs, proteins, peptides, or synthetic drugs, and the nucleic acid drugs further comprise plasmid DNA, antisense oligonucleotide, and RNAi. In the carrier composition, the drug and the cationic lipid have a weight ratio of about 1:4˜1:12, preferably 1:6.

The lipid based particle has a particle size of about 35˜95 nm, a zeta potential of about −10˜10 mV, an encapsulation efficiency of about 85˜100%, a drug release rate of about 60˜70% at pH4˜5, and the remained activity in serum is about 60˜100%.

Additionally, a ligand can be conjugated onto the surface of lipid based particle to recognize the receptor in a target cell. The lipid carrier with the ligand has a transfection efficiency exceeding 10 times that of a lipid carrier without a ligand.

The invention also provides a method of preparing the lipid carrier. First, a cationic lipid, a cholesterol, a neutral phospholipid, a neutral lipid, ethanol, and water are mixed to form a lipid solution, wherein the cationic lipid is about 100 parts by weight, the cholesterol is about 25˜100 parts by weight, the neutral phospholipid is about 25˜100 parts by weight, and the neutral lipid is about 25˜150 parts by weight. Next, a drug-containing solution is added to the lipid solution to form a suspension comprising a plurality of lipid based particles, wherein the drug is encapsulated into the lipid based particles. Finally, the suspension is heated to form a lipid carrier.

The solution is heated to about 50˜70° C., preferably 65° C. The ethanol and water have a volume ratio of about 3:7˜5:5, preferably 4:6.

The invention provides lipid based particles with uniform particle size, high encapsulation efficiency, and high serum resistance by using the specific ratio of carrier composition, ethanol/water ratios, drug/cationic lipid ratios, and heating conditions. After the lipid based particle entering the cell, the carrier will release the drug in a weak acidic lysosome in pH4˜5 due to its pH sensitivity, without decomposing by lysosome enzymes, could significantly increasing transfection efficiency of genes.

### EXAMPLES

Preparation of Lipid Carrier

First, 0.05 mL ethanol was added to a 1.5 mL centrifuge tube. Next, 0.1 mg cationic DOTAP, 0.05 mg cholesterol, 0.05 mg HSPC, and 0.065 mg DSPE-PEG were dissolved in the ethanol with water bath at 65° C. Deionized water was then added to the solution until the volume of 0.095 mL and mixed completely to form a lipid solution.

Next, 0.005 mL oligonucleotide-containing solution (10 mg/mL) was added to the lipid solution to form a mixture comprising a plurality of lipid based particles, wherein oligonucleotide was encapsulated into the lipid based particles. Finally, 0.1 mL PBS solution (pH7.4) was added to the centrifuge tube with a water bath at 65° C. for 10 min to form lipid carriers.

Measurement of Size and Zeta Potential of Lipid Based Particle

The size of the lipid based particle was measured by Coulter N4 Plus Submicron Particle Sizer (Miami, Fla.) and the zeta potential thereof was measured by ZetaPlus Zeta Potential Analyzer (Brookhaven Instruments Corporation, Holtsville, N.Y.). The measured diameter was 72.0±22.5 nm and the zeta potential was −0.302 mV.

Measurement of Encapsulation Efficiency

Encapsulated oligonucleotides and free oligonucleotides were separated by a chromatography column. The encapsulation efficiency was then measured. The calculation is illustrated in the following.

Encapsulation efficiency (%)=(total amount of oligonucleotide—the amount of free oligonucleotide)/the amount of total oligonucleotide×100%

The encapsulation efficiency of the carrier was calculated as 95±5%

Measurement of Drug Release Rate

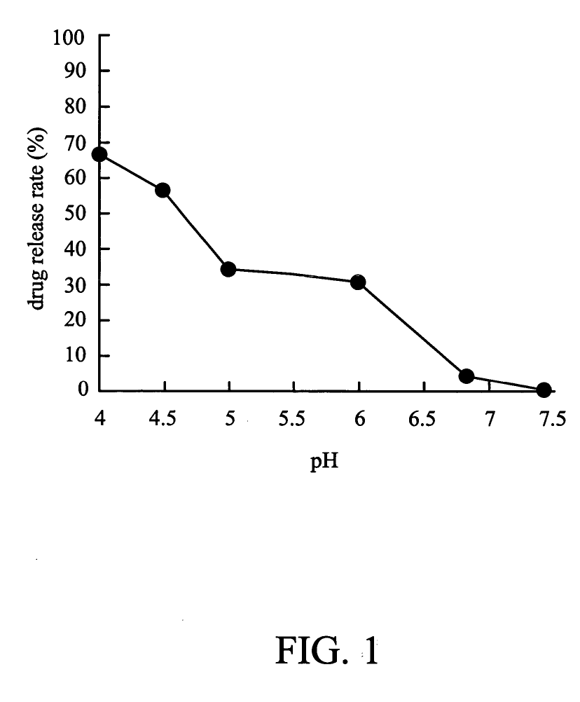

0.2 mL carrier solution was mixed with 100 mM buffer solutions (pH6.8, pH6, pH5.5, pH5, pH4.5, pH4), respectively, with water bath at 37° C. for 5 min. After the lipid solution was separated by a Sepharose CL 4B chromatography column, the release rate of oligonucleotide was measured. Referring to FIG. 1, the carrier has a drug release rate of about 60˜70% at pH4˜5.

Measurement of Serum Resistance

PC14PE6 cells were cultured on a 1 cm² glass slide. The slide contained 2×10⁴ cells after culturing. Serum resistance testing was performed as the following steps. First, the cells and carriers encapsulated oligonucleotide were cultured in two mediums at 37° C. for 4 hours, respectively, wherein one medium contained 10% serum and another one without serum. The cells were washed by PBS solution three times and were dissolved by mixing with another portion of PBS solution containing 1% Triton X-100 for 1 hour. Finally, the exuded cytosol was analyzed by a fluorescent analyzer (Ex: 494 nm, Em: 519 nm). According to the analytical results, the carrier maintained its activity within 60˜100% in serum.

Measurement of Transfection Efficiency of Carrier with Ligand

PC14PE6 cells were cultured in a 24 wells night over. Each well contained 5×10⁴ cells after culturing. Transfection efficiency was tested as follows. Cells and carriers with or without tamoxifen ligands were cultured in two mediums at 37° C. for 4 hours, respectively, wherein one medium contained 10% serum and another one contained no serum. Next, the cells were washed by PBS solution and dissolved by mixing with another portion of PBS solution containing 1% Triton X-100 for 1 hour. Finally, the exuded cytosol was analyzed by a fluorescent analyzer (Ex: 494 nm, Em: 519 nm).

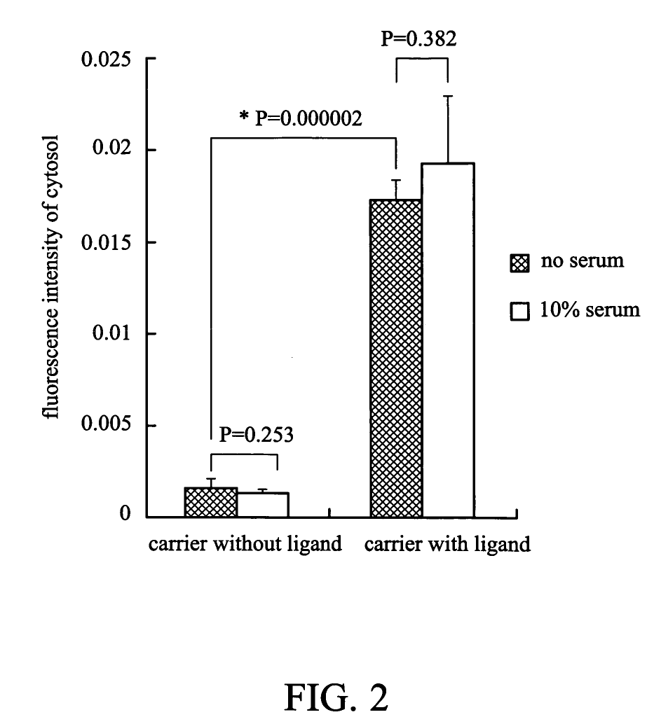

Referring to FIG. 2, the results indicate that the carrier with a ligand has a transfection efficiency exceeding 10 times that of the carrier without a ligand. Additionally, serum resistance of the lipid carrier provided by the invention has been proven because the test results under serum or without serum are the same.

While the invention has been described by way of example and in terms of preferred embodiment, it is to be understood that the invention is not limited thereto. To the contrary, it is intended to cover various modifications and similar arrangements (as would be apparent to those skilled in the art). Therefore, the scope of the appended claims should be accorded the broadest interpretation so as to encompass all such modifications and similar arrangements.

What is claimed is:

1. A lipid carrier, comprising:
a lipid based particle comprising a cationic lipid, a cholesterol, a neutral phospholipid, and a neutral lipid, wherein the cationic lipid is about 100 parts by weight, the cholesterol is about 25˜100 parts by weight, the neutral phospholipid is about 25˜100 parts by weight, and the neutral lipid is about 25˜150 parts by weight.

2. The lipid carrier as claimed in claim 1, wherein the cationic lipid comprises 1,2-dioleoyloxy-3-(trimethylamino)propane (DOTAP), N-[1-(2,3-ditetradecyloxy)propyl]-N,N-dimethyl-N-hydroxyethylammonium bromide (DMRIE), N-[1-(2,3-dioleyloxy)propyl]-N,N-dimethyl-N-hydroxyethylammonium bromide (DORIE), N-[1-(2,3-dioleyloxy)propyl]-N,N,N-trimethylammonium chloride (DOTMA), 3β-[N-(N′,N′-dimethylaminoethane)carbamyl]cholesterol (DC-Chol), or dimethyldioctadecylammonium (DDAB).

3. The lipid carrier as claimed in claim 1, wherein the neutral phospholipid comprises phosphatidyl choline (PC) or phosphatidyl ethanolamine (PE).

4. The lipid carrier as claimed in claim 3, wherein the phosphatidyl choline comprises hydrogenated soy phosphatidyl choline (HSPC).

5. The lipid carrier as claimed in claim 1, wherein the neutral lipid comprises distearoylphosphatidylethanolamine-polyethyleneglycol (DSPE-PEG).

6. The lipid carrier as claimed in claim 1, wherein the cationic lipid is about 100 parts by weight, the cholesterol is about 50 parts by weight, the neutral phospholipid is about 50 parts by weight, and the neutral lipid is about 65 parts by weight.

7. The lipid carrier as claimed in claim 1, further comprising a drug encapsulated into the lipid based particle.

8. The lipid carrier as claimed in claim 7, wherein the drug comprises nucleic acid drugs, protein drugs, peptide drugs, or synthetic drugs.

9. The lipid carrier as claimed in claim 8, wherein the nucleic acid drugs comprise plasmid DNA, antisense oligonucleotide, or RNAi.

10. The lipid carrier as claimed in claim 7, wherein the drug and the cationic lipid have a weight ratio of about 1:4˜1:12.

11. The lipid carrier as claimed in claim 7, wherein the drug and the cationic lipid have a weight ratio of about 1:6.

12. The lipid carrier as claimed in claim 1, wherein the lipid based particle has a diameter of about 35˜95 nm.

13. The lipid carrier as claimed in claim 1, wherein the lipid based particle has a zeta potential of about −10˜10 mV.

14. The lipid carrier as claimed in claim 1, wherein the lipid based particle has an encapsulation efficiency of about 85˜100%.

15. The lipid carrier as claimed in claim 7, wherein the lipid carrier has a drug release rate of about 60˜70% at pH4˜5.

16. The lipid carrier as claimed in claim 1, wherein the lipid carrier has an activity of about 60˜100% in serum.

17. The lipid carrier as claimed in claim 1, further comprising a ligand grafted onto the lipid based particle surface.

18. The lipid carrier as claimed in claim 17, wherein the ligand recognizes a target cell of a subject.

19. The lipid carrier as claimed in claim 17, wherein the lipid carrier with the ligand has transfection efficiency exceeding 10 times that of a lipid carrier without a ligand.

20. A method of preparing a lipid carrier, comprising:
mixing a cationic lipid, a cholesterol, a neutral phospholipid, a neutral lipid, ethanol, and water to form a lipid solution, wherein the cationic lipid is about 100 parts by weight, the cholesterol is about 25˜100 parts by weight, the neutral phospholipid is about 25˜100 parts by weight, and the neutral lipid is about 25˜150 parts by weight;
adding a drug-containing solution to the lipid solution to form a solution comprising a plurality of lipid based particle, wherein the drug is encapsulated into the lipid based particle; and
heating the solution to form a lipid carrier.

21. The method as claimed in claim 20, wherein the cationic lipid comprises 1,2-dioleoyloxy-3-(trimethylamino)propane (DOTAP), N-[1-(2,3-ditetradecyloxy)propyl]-N,N-dimethyl-N-hydroxyethylammonium bromide (DMRIE), N-[1-(2,3-dioleyloxy)propyl]-N,N-dimethyl-N-hydroxyethylammonium bromide (DORIE), N-[1-(2,3-dioleyloxy)propyl]-N,N,N-trimethylammonium chloride (DOTMA), 3β-[N-(N′, N′-dimethylaminoethane)carbamyl]cholesterol (DC-Chol), or dimethyldioctadecylammonium (DDAB).

22. The method as claimed in claim 20, wherein the neutral phospholipid comprises phosphatidyl choline (PC) or phosphatidyl ethanolamine (PE).

23. The method as claimed in claim 22, wherein the phosphatidyl choline comprises hydrogenated soy phosphatidyl choline (HSPC).

24. The method as claimed in claim 20, wherein the neutral lipid comprises distearoylphosphatidylethanolamine-polyethyleneglycol (DSPE-PEG).

25. The method as claimed in claim 20, wherein the cationic lipid is about 100 parts by weight, the cholesterol is about 50 parts by weight, the neutral phospholipid is about 50 parts by weight, and the neutral lipid is about 65 parts by weight.

26. The method as claimed in claim 20, wherein the ethanol and water have a volume ratio of about 3:7˜5:5.

27. The method as claimed in claim 20, wherein the ethanol and water have a volume ratio of about 4:6.

28. The method as claimed in claim 20, wherein the drug comprises nucleic acid drugs, protein drugs, peptide drugs, or synthetic drugs.

29. The method as claimed in claim 28, wherein the nucleic acid drugs comprise plasmid DNA, antisense oligonucleotide, or RNAi.

30. The method as claimed in claim 20, wherein the drug and the cationic lipid have a weight ratio of about 1:4˜1:12.

31. The method as claimed in claim 20, wherein the drug and the cationic lipid have a weight ratio of about 1:6.

32. The method as claimed in claim 20, wherein the solution is heated to about 50˜70° C.

33. The method as claimed in claim 20, wherein the solution is heated to about 65° C.

34. The method as claimed in claim 20, wherein the lipid based particle has a diameter of about 35˜95 nm.

35. The method as claimed in claim 20, wherein the lipid based particle has a zeta potential of about −10˜10 mV.

36. The method as claimed in claim 20, wherein the lipid based particle has a encapsulation efficiency of about 85˜100%.

37. The method as claimed in claim 20, wherein the lipid carrier has a drug release rate of about 60˜70% at pH4˜5.

38. The method as claimed in claim 20, wherein the lipid carrier has an activity of about 60˜100% in serum.

39. The method as claimed in claim 20, further comprising a ligand grafted onto the lipid based particle surface.

40. The method as claimed in claim 39, wherein the ligand recognizes a target cell of a subject.

41. The method as claimed in claim 39, wherein the lipid carrier with the ligand has transfection efficiency exceeding 10 times that of a lipid carrier without a ligand.
